(19) United States
(12) Patent Application Publication
(10) Pub. No.: US 2023/0097882 A1
(43) Pub. Date: Mar. 30, 2023

(54) ADDITIVE MANUFACTURED FOOTSHELLS AND FOOTSHELL COMPONENTS

(71) Applicant: Otto Bock HealthCare LP, Austin, TX (US)

(72) Inventors: Florian Wilhelm Sebastian Poser, Salt Lake City, UT (US); Vaughn Roy Anderson, Highland, UT (US); Charles C. Polta, Cottonwood Heights, UT (US); Vijay Morampudi, South Jordan, UT (US); Randy Hall, Salt Lake City, UT (US); Douglas E. Rush, Salt Lake City, UT (US); Jules Revais, Seyssinet-Pariset (FR); Marc Souply, VIZILLE (FR); Léna Oberson, Grenoble (FR); Christian Daur, Berlin (DE)

(73) Assignee: Otto Bock HealthCare LP, Austin, TX (US)

(21) Appl. No.: 17/901,763

(22) Filed: Sep. 1, 2022

Related U.S. Application Data
(60) Provisional application No. 63/239,855, filed on Sep. 1, 2021.

(51) Int. Cl. A61F 2/66; A61F 2/50

(52) U.S. Cl. CPC A61F 2/66; A61F 2/5044; A61F 2002/665

(57) ABSTRACT

A prosthetic device includes a footshell having a first portion and a second portion. The first portion has a first portion polymer lattice structure and the second portion has a second portion polymer lattice structure. The first portion polymer lattice structure has a first set of mechanical properties and the second portion polymer lattice structure has a second set of mechanical properties different from the first set of mechanical properties. The footshell also includes a sole attached to a bottom of the first portion and the second portion, wherein the sole is formed of a solid material.

### CROSS REFERENCE TO RELATED APPLICATIONS

This application claims the benefit of and priority to U.S. Provisional Patent Application No. 63/239,855, filed Sep. 1, 2021, which is hereby incorporated by reference in its entirety.

### TECHNICAL FIELD

The present disclosure relates generally to prosthetic devices, and more particularly to prosthetic feet, footshells, and footshell components, and to related methods for making footshells and footshell components.

### BACKGROUND

It is highly desirable that prosthetics are lightweight and strong to enable increased user mobility and increased durability of the prosthetic. Specifically, foot prosthetics with decreased weight and increased strength enable increased mobility of the user and extended life of the foot prosthetic. Current prosthetic feet are typically made of rigid carbon fiber composite materials or thick thermoplastic materials. Prosthetic feet are typically comprised of two elements, a strong structural component or components to facilitate the transfer of forces from the ground to the residual limb, and a cosmetic cover or footshell is used to approximate the look of a human foot and provide a functional fit between the structural components and a shoe. Prosthetic feet are typically formed using unidirectional fiber reinforced plastic materials to minimize weight and provide adequate strength. Achieving adequate strength is a design challenge and hence a prosthetic foot may be stiffer than desired to withstand worst case loading events. An amputee may engage in a variety of activities, and a typical prosthetic foot does not adjust or adapt the foot stiffness to different activities. In addition, the weight and activity level of an amputee may vary over time.

For the foregoing reasons, there is a need to provide improved prosthetic feet that are optimized for various activities, activity levels, and weights of the user.

### SUMMARY

One aspect of the present disclosure relates to a prosthetic device which includes a footshell having a lattice structure.

Another aspect of the present disclosure relates to a prosthetic device which includes a footshell having a first portion and a second portion. The first portion has a first portion polymer lattice structure and the second portion has a second portion polymer lattice structure. The first portion polymer lattice structure has a first set of mechanical properties and the second portion polymer lattice structure has a second set of mechanical properties different from the first set of mechanical properties. The footshell also includes a sole attached to a bottom of the first portion and the second portion, wherein the sole is formed of a solid material.

Another aspect of the present disclosure relates to a prosthetic device including a footshell including a first portion and a second portion. The first portion having a first portion polymer lattice structure and the second portion is formed of a solid material. The second portion is a sole portion and the sole portion is located below a distalmost surface of a prosthetic foot.

The present disclosure is also directed to a method of manufacturing a footshell. The method may include forming a first portion of the prosthetic device with a first lattice structure and forming a second portion of the prosthetic device with a second lattice structure having at least one of a different property than that of the first lattice structure. The first and second lattice structures are formed as a continuous, integral structure using an additive manufacturing process.

The foregoing has outlined rather broadly the features and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of the present disclosure. Such equivalent constructions do not depart from the spirit and scope of the appended claims. Features which are believed to be characteristic of the concepts disclosed herein, both as to their organization and method of operation, together with associated advantages will be better understood from the following description when considered in connection with the accompanying figures. Each of the figures is provided for the purpose of illustration and description only, and not as a definition of the limits of the claims.

### BRIEF DESCRIPTION OF THE DRAWINGS

A further understanding of the nature and advantages of the embodiments may be realized by reference to the following drawings. In the appended figures, similar components or features may have the same reference label.

FIG. 1 is a cross-sectional side view of an example of a 3D printed prosthetic device in accordance with the present disclosure.

FIG. 2 is a perspective view of another example of a 3D printed prosthetic device in accordance with the present disclosure.

FIG. 3 is a cross-sectional side view of another example of a 3D printed prosthetic device in accordance with the present disclosure.

FIGS. 4A, 4B and 4C are cross-sectional side views of another example of a 3D printed prosthetic device in accordance with the present disclosure.

FIG. 5 is a cross-sectional side view of another example of a 3D printed prosthetic device in accordance with the present disclosure.

FIG. 6 is a cross-sectional side view of another example of a 3D printed prosthetic device in accordance with the present disclosure.

FIG. 7 is a cross-sectional side view of another example of a 3D printed prosthetic device in accordance with the present disclosure.

FIG. 8 is a cross-sectional side view of a portion of the 3D printed prosthetic device illustrated in FIG. 7 in accordance with the present disclosure.

FIG. 9 is a cross-sectional side view of another example of a 3D printed prosthetic device in accordance with the present disclosure.

FIG. 10 is a cross-sectional side view of another example of a 3D printed prosthetic device in accordance with the present disclosure.

FIGS. 11A and 11 B are perspective views of another example of a 3D printed prosthetic device in accordance with the present disclosure.

FIGS. 12A-B are a cross-sectional side views of another example of a 3D printed prosthetic device in accordance with the present disclosure.

FIGS. 13A and 13B are each cross-sectional side views of another example of a 3D printed prosthetic device in accordance with the present disclosure.

FIG. 14 is a cross-sectional side view of a portion of the 3D printed prosthetic device illustrated in FIGS. 13A and 13B in accordance with the present disclosure.

FIGS. 15A and 15B are cross-sectional side views of a portion of the 3D printed prosthetic device illustrated in FIGS. 13A, 13B and 14 in accordance with the present disclosure.

FIG. 16A is a perspective view of another example of a 3D printed prosthetic device in accordance with the present disclosure.

FIG. 16B is a cross-sectional side view of a portion of the 3D printed prosthetic device illustrated in FIG. 16A in accordance with the present disclosure.

FIG. 17 is a perspective view of another example of a 3D printed prosthetic device in accordance with the present disclosure.

FIG. 18 is a cross-sectional side view of a portion of the 3D printed prosthetic device illustrated in FIG. 17 in accordance with the present disclosure.

FIG. 19 is a perspective view of another example of a 3D printed prosthetic device in accordance with the present disclosure.

FIG. 20 is a chart showing lattice structures which mimic naturally-occurring atomic structures for use in 3D printed prosthetic devices of the present disclosure.

FIGS. 21A, 21B, 21C, 21D, 21E and 21F are perspective views of exemplary lattice structures for use with the 3D printed prosthetic devices disclosed herein.

FIG. 22 is a flow diagram illustrating an exemplary method in accordance with the present disclosure.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

The present disclosure is generally directed to prosthetic devices, and more particularly relates to footshells and footshell components and related methods for the manufacture of footshells and footshell components. The footshells and footshell components disclosed herein may be formed concurrently and together as a unitary, integral structure. In some embodiments, the footshells, footshell components, or combinations thereof may be formed using an additive manufacturing process, such as a 3D printing process. Various materials may be used to form the footshells and footshell components disclosed herein. In some embodiments, the same material may be used to form both the footshells and footshell components, while in others, more than one or a variety of materials may be used. Similarly, the lattice structure used in the footshells and footshell components may be different in different regions of the footshells and/or footshell components to provide different amounts of stiffness, airflow, and other properties to the device.

Additive manufacturing, also known as 3D printing, utilizes a variety of technologies to create a structure. One technology uses focused laser energy to create chemical reactions which cure liquid polymer in a bath layer by layer. Another method extrudes melted material layer by layer. These methods all add material to a part or structure in thin layers, typically, without limitation, from about 0.003 inches to about 0.030 inches of thickness per layer. Each layer of new material being applied bonds to the existing layer by means of the melted entanglement of polymer chains or by chemical reactions or some combination of the two.

Additive manufacturing methods have the ability to create sparse structures. These structures may be similar to a three-dimensional truss structure in which connected rods or beams of material produce efficient, light-weight structures. By altering the angles, thickness, and/or frequency of the individual rods or beams it is possible to control the mechanical response of the resulting structure.

The additive nature of the technology may provide the ability to create complex geometry and other desirable properties in a cost-effective manner. Many of these geometric shapes cannot be created using other known manufacturing methods. For example, footshells with lattice type structures can be optimized for performance by varying the density or the geometry of the lattice. The footshell can be customized in a variety of ways by merely changing the digital 3D model used to create the footshell. Each footshell can be customized to meet the user's specific needs with little impact on manufacturing costs.

The most common 3D printing materials used today are polymers, which are an acceptable material for footshell and footshell component constructions. Footshells and footshell components created using 3D printing can have wide temperature compatibility, variable strength and/or stiffness, and be biocompatible. Since components are constructed one thin layer at a time, normal design restrictions such as angles and contour, lattice density, surface characteristics, smoothness, undercuts, and cavities do not necessarily apply to 3D printed articles. Not only does 3D printing allow more design freedom, it also allows complete customization of designs. Current additive manufacturing technologies may be perfectly suited in many instances for producing custom footshells, footshell components, and footshell/footshell component combinations.

An example of such customization relates to a footshell that is specifically designed for the individual recipient. A 3D printed footshell would improve the ability to provide footshells for maximum comfort. In addition to providing an accurate fit, a 3D printed footshell can be designed such that the structural compression stiffness, bending stiffness, and heat conduction properties can vary continuously along any dimension. Localized areas can be made softer or harder, and/or be made more rigid or more flexible. Current footshell designs are generally based upon a monolithic structure and properties.

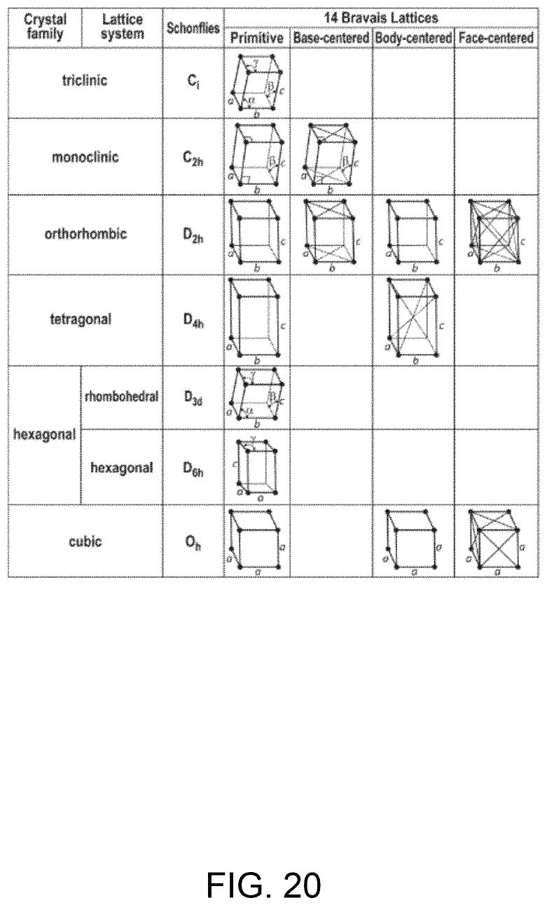

Creating a lightweight, porous, and/or variable stiffness structure by additive manufacturing techniques may utilize a repeating cell structure. Cell structures can mimic naturally occurring atomic structures such as cubic, tetragonal, orthorhombic, rhombohedral, monoclinic, triclinic, including body centered, face centered, and base centered variations of these atomic cells shown in FIG. 20. The atom positions in such cell structure may represent connection points or nodes between multiple individual truss members which may consists of rods or beams of material. Another naturally occurring cell structure is honeycomb. However, 3D Printed cell structures do not need to mimic naturally occurring structures. For example, additive manufacturing can be used to create a series of interconnected coil springs. By altering the angles, thickness, and/or frequency of the individual rods or beams used to create an additive manufactured cell structure it is possible to control the mechanical response of the resulting structure. The repeating cell structure may vary in that the distance between nodes or the thickness or diameter of the rods in one or more dimensions. The cells may become progressively larger or smaller and/or the nodes and/or rods may become larger or smaller in one or more part dimensions, such as though-the thickness, or in a radial direction, or in a proximal-distal direction. The dimensions of the cells, or nodes, or rods may be continuously variable.

3D Printing may be advantageously used to create simpler structures than three-dimensional rod and node lattice structures. For example, shell type structures with perforations may be difficult and expensive to create using traditional manufacturing techniques. Molding perforations into a part typically requires a retractable insert in the mold for each perforation. If the shell structure is curvilinear, cutting perforations is also difficult as the cutting tools must follow the contour of the surface of the shell, for example in prosthetic footshells. In addition, cutting perforations typically leaves an undesirable finish on the surfaces which are cut. For the purposes of this disclosure, a shell structure with perforations is defined as a two-dimensional (2D) lattice.

Various additive manufacturing technologies are available and those applicable to polymer materials include Powder Bed Fusion (PBF), Vat Photopolymerization, Material Extrusion, and Material Jetting. Powder bed fusion includes Selective Laser Melting (SLM), selective laser sintering (SLS) and selective heat sintering (SHS). PBF involves spreading a thin layer of powdered material on a surface and then melting the powder to fuse the particles. Thermal energy in the form of a laser or a heated print head may provide the required melt energy.

Vat photopolymerization utilizes liquid photopolymer resin bath and a laser to create localized chemical reaction resulting in a polymer structure. Stereolithography (SLA) is the most common method with variations including Direct Light Processing (DLP) which uses microscopic mirrors to project the laser at multiple locations to eliminate the necessity of tracing each layer with the laser, and Continuous Direct Light Processing (CDLP) adds a continuously moving build platform. DLP and CDLP result in faster part build times. Vat Photopolymerization may be a preferred method to create parts made with elastomeric materials.

Material Extrusion consists of Fused Deposition Modeling (FDM) or Fused Filament Fabrication (FFF). In this process, a small thermoplastic filament is extruded through a heated nozzle and melt bonded to the previous layer of deposited material.

Material Jetting utilizes tiny print head nozzles to dispense tiny droplets of photopolymer layer by layer. UV light is used to cure the droplets. This technique is similar to the process used in ink jet printing.

Another additive manufacturing is Rapid Liquid Printing. Rapid Liquid Printing extrudes thermoset material into a bath of viscous gel. The gel supports the printed structure until the thermoset material cures and becomes self-supporting. Rapid Liquid Printing provides high print speeds and surface finish quality. Other similar processes may exist which would be suitable for use with the present disclosure.

Footshells are typically constructed of elastomeric polymer material because the flexibility, wear resistance, and durability are well suited to this application. Prosthetic feet and footshells often are not optimal over the entire range of potential uses. For example, a foot and footshell that is comfortable for walking may not be an appropriate foot and footshell for running. A 3D printed footshell could be formed in such a way to accommodate for different uses, an activity level which changes over time, or increasing or decreasing amputee weight by altering the geometry of the lattice of different regions of the footshell. In addition, footshells are less expensive and easier to replace than an entire prosthetic foot. Replacing a footshell may be performed by an amputee without the assistance of a prosthetist. Hence utilizing an advanced footshell design to alter and/or optimize the performance a prosthetic foot and footshell combination is highly desirable.

Additionally, 3D printed structures may offer the unique ability to separate and disconnect properties which have historically been considered inherent material properties. For example, as a material is stretched in one direction/dimension the material contracts in the other two dimensions (i.e., the Poisson's effect). A 3D printed structure having a lattice structure has a response dependent on the geometry of the lattice, not necessarily on the direction a force is applied. This is different than the response of the material used to create the lattice structure. Additive manufacturing can be used to create auxetic material structures with a negative Poisson's ratio, which results in material expansion in one or more directions perpendicular to an applied tensile force and contraction in one or more directions perpendicular to an applied compressive force. Auxetic material structures present a method to address unique problems experienced by amputees and may be used to improve fit or function in a footshell

Additive manufactured structures can be made using a variety of materials, which include thermoset polymers, thermoplastic polymers, metals, and fiber reinforced composites. Elastomers are commonly defined as rubber-like materials. Elastomers can be defined by hardness, maximum elongation, modulus, Poisson's ratio, or glass transition temperature and by combinations of these properties. Elastomeric materials are available in a wide range of hardnesses or stiffnesses ranging from hard elastomers with a Young's modulus of 500,000 psi to very soft elastomers with a secant modulus of 50 psi at 100% elongation. As elastomeric materials become harder their properties become more linear elastic and follow Hooke' s Law. Hence it can be difficult to differentiate an elastomeric material from a non-elastomeric material.

Elastomers are difficult to characterize because they have viscoelastic properties. The mechanical response of an elastomer is partially elastic and partially viscoelastic. A viscoelastic response may be characterized by a linear spring in parallel with a dashpot. A dashpot is a damping device such as a hydraulic cylinder. The spring provides resistance to compression and extension, while the dashpot slows both the compression and extension, and the recovery from compression and extension. Hence the mechanical response is time dependent.

For the purposes of this application, an elastomer is defined as a material which when stretched to 10% elongation at room temperature will recover at least 70% of the deformation within 10 seconds when the stretching load is removed.

Indentation hardness testing is a method of characterizing the stiffness of a material. This type of test may also be known as a durometer, indentation or durometer hardness test. Hardness testing can be performed on a wide range of materials, from the hardest steels to soft elastomers. ASTM D 2240, DIN 53505, and ISO R/868 are comparable methods used for testing both soft elastomers and hard plastic materials and the results are stated on the Shore scale.

The commonly used Shore hardness scales, in increasing order of hardness, are 000-S, 000, 00, 0, and A-D. The Shore D, A and 00 scales are most commonly used because these 3 scales result in a functional continuum for hardnesses in most situations. Shore-OO 63 is approximately equivalent to Shore-A 20 and Shore-A 73 is approximately equivalent to Shore-D 20. Typical commonly known material hardnesses are Shore-OO 20 for chewing gum, Shore-A 25 for a rubber band, Shore-A 70 for tire tread, and Shore-D 70-90 for rigid plastics like nylon and polyethylene. Some structural plastics and composite materials have hardnesses which exceed the Shore scales. Shore-000-S and Shore-000 scales are typically used for soft foams or sponges.

Most currently available prosthetic footshells have a hardness of approximately Shore-A 60, although values from around Shore-A 40 to Shore-A 90 may be in use. One of the advantages of additive manufacturing is the ability to utilize a lattice structure, sometimes known as a sparse structure. As the void content of a material increases, for example in foams, the material becomes softer. Hence, attaining the desired stiffness when utilizing a lattice structure in a prosthetic footshell there may be a need to utilize a stiffer raw material, as compared to monolithic footshell. The Shore hardness of the material used in an additive manufactured footshell utilizing a lattice structure may extend into the Shore D scale.

When combining a footshell with footshell components into a single, unitary structure, the stiffness of the material used may vary across specific footshell dimensions to provide the required stiffness and strength to resist reaction forces and yet allow for control of the footshell. This may be achieved by changing the stiffness of the material, by changing the density of the lattice structure, by changing the geometry of the lattice, or all of the above.

3D printed footshells and footshell components provide the option of changing the lattice structure to make sections harder or softer and to control the mechanical response of the footshell in different directions. The cell structure can be altered such that the stiffness changes in one direction, for example, while leaving the stiffness in other directions the same, or even increasing the stiffness in the other directions. With the option of changing material lattice modulus or stiffness, enhanced stability, security, fit comfort, and performance may be achieved.

In one embodiment, the material used to create a lattice structure varies in hardness through the thickness. Elastomeric materials are available in different hardnesses and these different hardness formulations may have similar atomic or chemical structures. As a result, materials from the same family of elastomeric product tend to bond to each other well. It is also possible to find materials from different manufacturers which bond well to each other. As a result, in some examples, the material used to form the footshell may be changed to a material of a different hardness during the additive manufacturing process from one region or segment of the footshell to another. If the two materials are compatible, a strong connection will typically occur at the interface between the two materials. In one example, this approach may provide a soft inner layer and a stiffer, stronger structure on the outer layers (or visa-versa) as part of a laminated lattice structure.

In another variation, a lattice footshell construction includes a trellis or web structure of polymeric material. This construction provides a porous structure, wherein air can flow freely through the footshell, decreasing the density and weight of the footshell. The decreased density and weight of the footshell may provide improved comfort as well as increased mobility. A measure of air permeability is rate of airflow passing perpendicularly through a known area under a prescribed air pressure differential between the two surfaces of a material. A common test for fabric materials is ASTM D737-96. This test method can be adapted to thicker materials.

In another embodiment, the footshell could be made as a single piece using a 3D printing process. An inner portion of the footshell may be relatively pliable and soft, and an outer surface of the footshell could be made more rigid so as to be supportive of the prosthesis and provide improved strength and/or wear resistance. Both the inner portion and outer surfaces of the footshell structure could be made with a plurality of void spaces to decrease density and weight.

Some advantages related to the 3D printed footshells disclosed herein include reduced density and weight and improved mobility from a lattice structure. Another advantage relates to the ability to customize properties of the footshell. For example, the footshell may have a customized hardness or softness in particular areas when using a single material. The footshell may have different hardness or softness in particular areas by using materials with different hardness or stiffness properties. The footshell may have sections with different rigidity and flexibility properties. The footshell may have variable compressible/expandable properties that allow for physical changes in a person's residual limb. Further, as mentioned above, the footshells may be formed as a single-piece, unitary device. In addition, a complete prosthetic foot or limb may be 3D printed, including the 3D printed footshells described herein.

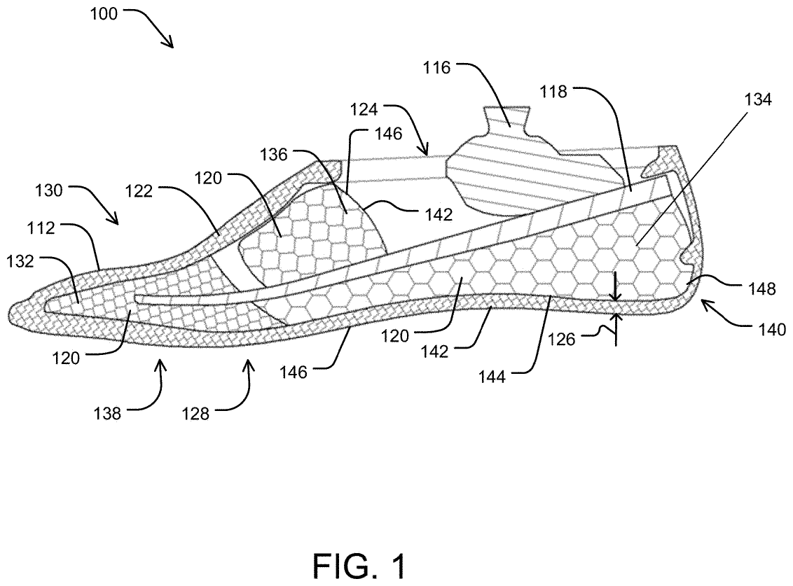

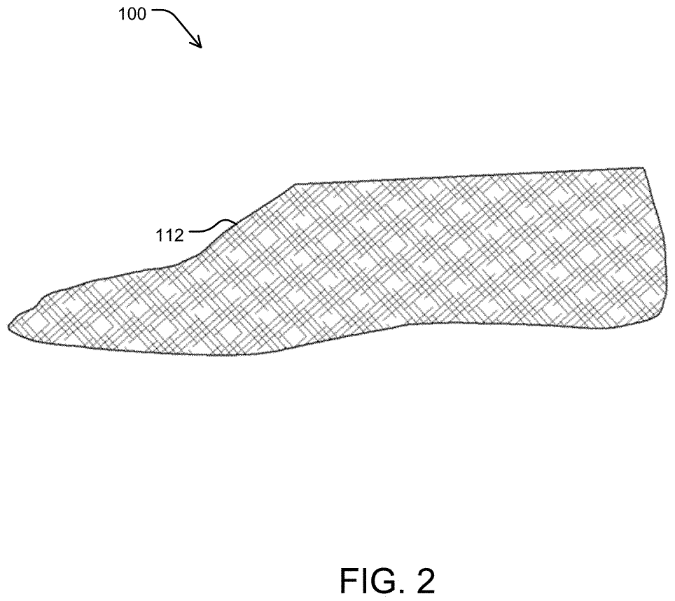

FIG. 1 illustrates a schematic cross section of an example of a 3D printed device 100. FIG. 2 illustrates a perspective view of a footshell 112 of the 3D printed device 100. Referring now to FIGS. 1 and 2, the example 3D printed device 100 is shown and described. The 3D printed device 100 includes the footshell 112 and may also include at least one internal component. In some embodiments, the footshell 112 may be formed as a unitary device without any separate components. In other embodiments, the footshell 112 and the internal components may be formed as separate components and assembled after the footshell 112 and the internal components are formed. The internal components may include an adapter 116, a spring 118, and one or more fillers 120. The spring and adapter are typical structural components of a prosthetic foot. In the illustrated embodiment, only the fillers 120 of the internal components are formed by an additive manufacturing process. In alternative embodiments, all the internal components are additively manufactured. The 3D printed device 100 is configured to be mounted to a limb (not shown), such as a residual limb remaining after an amputation. An example of a residual limb may be a residual limb associated with a below-the-knee amputation.

One or more of the footshell 112 and the internal components may be formed or manufactured via an additive manufacturing process such as 3D printing, which forms the footshell 112 and the internal components from a three-dimensional lattice network. More specifically, in the embodiment illustrated in FIG. 2, the footshell 112 is formed or manufactured via an additive manufacturing process, or 3D printing, which forms the footshell 112 from a three-dimensional lattice network. Manufacturing the footshell 112 using an additive manufacturing process may reduce costs because the additive manufacturing process does not require a mold into which a polymer is injected and because a lattice requires less material than a solid part. Thus, manufacturing the footshell 112 using an additive manufacturing process may significantly reduce the tooling cost of producing the footshell 112. Additionally, manufacturing the footshell 112 using an additive manufacturing process enables the footshell 112 to be customized to the need of the user.

The footshells such as 112 described herein are manufactured using additive manufacturing process to form a lattice structure. The lattice structure enables the weight of the footshell 112 to be reduced and the design of the footshell 112 to be optimized for a user and/or a desired use of the footshell 112. Such additively manufactured footshells 112 may also be customized to accommodate unusual or infrequently needed sizes for users, making it financially feasible to create and supply footshells to meet a wider variety of customer needs. Varying the design of the lattice structure used to make up the footshell 112 also enables the weight and the mechanical properties of the footshell 112 to be optimized. The optimized mechanical properties may include stiffness, resiliency, strength, and durability. The optimized mechanical properties may also include the liquid/moisture transport properties of the footshell 112 and friction properties of the footshell 112 against a surface or surfaces. The physical properties of the footshells 112 described herein may be adjusted by altering the design parameters of the lattice structure and the material or materials used to create the lattice structure.

Additionally, the internal components described herein may be manufactured using additive manufacturing processes to form them using lattice structures. As with the footshell 112, use of lattice structures in at least one of the internal components enables the weight of at least one of the internal components to be reduced and for the design of at least one of the internal components to be optimized. Varying the design of the lattice structure enables the weight and the mechanical properties of at least one of the internal components to be optimized. The optimized mechanical properties may include stiffness, resiliency, strength, and durability. The optimized mechanical properties may also include the liquid/moisture transport properties of at least one of the internal components and friction properties of at least one of the internal components against the other internal components and/or the footshell 112. The physical properties of at least one of the internal components described herein may be adjusted by altering the design parameters of the lattice structure and the material or materials used to create the lattice structure.

In the embodiment illustrated in FIGS. 1 and 2, the footshell 112 includes an outer shell 122 that defines a footshell cavity 124. Specifically, in the illustrated embodiment, the footshell 112 is formed using the additive manufacturing process such that the footshell 112 is substantially hollow to reduce and/or optimized the weight of the footshell 112. More specifically, the outer shell 122 is formed using the additive manufacturing process such that the footshell cavity 124 is formed within the outer shell 122. The outer shell 122 has an outer shell thickness 126 that may be optimized or varied to optimize or tune the weight and mechanical properties of the footshell 112. Specifically, the outer shell thickness 126 may be the same or may vary throughout the footshell 112 to optimize or tune the weight and mechanical properties of the footshell 112.

Additionally, the material of the outer shell 122 may also be varied to optimize or tune the weight and mechanical properties of the footshell 112. For example, a first portion of the outer shell 122 may be formed of a first material having a first density and a first set of mechanical properties and a second portion of the outer shell 122 may be formed of a second material having a second density and a second set of mechanical properties. Specifically, a plantar portion 128 of the footshell 112 may be formed of a denser, harder material than a dorsal portion 130 of the footshell 112 because the plantar portion 128 may contact the ground and support the user. Accordingly, the material of the outer shell 122 may also be varied to optimize or tune the weight and mechanical properties of the footshell 112. In alternative embodiments, the footshell 112 may be entirely formed of the same material.

As shown in FIG. 1, the 3D printed device 100 is shown to house an adapter 116, spring 118, and one or more fillers 120. In the illustrated embodiment, the 3D printed device 100 includes a plurality of fillers 120. Specifically, the fillers 120 include a toe wedge 132, a heel wedge 134, and a shoe filler 136 arranged with the adapter 116 and the spring 118 in the footshell cavity 124. The fillers 120 are configured to provide additional support and optimization of the footshell 112. Specifically, the fillers 120 may be inserted into the footshell cavity 124 to optimize the fit of the spring 118 and adapter 116 within the footshell 112. As a result, the outer shell 122 may be formed of a first material having a first density and a first set of mechanical properties and the fillers 120 may be formed of a second material having a second density and a second set of mechanical properties. The first material may be harder and denser than the second material because the footshell 120 may contact the ground and support the user while second material may have greater stiffness than the first material to support the user during the gait cycle.

The fillers 120 and the outer shell 122 are formed of a lattice structure such that the fillers 120 and the outer shell 122 have cushioning or dampening characteristics. The cushioning or dampening properties of the fillers 120 and the outer shell 122 may be customized for the user's weight and activity level to provide a more comfortable user experience. In some embodiments, the fillers 120 and the outer shell 122 may be changed as the user's mobility changes over time to accommodate increased or decreased activity without incurring the expense of new structural components (i.e. an entire foot). In certain situations, one or more of the fillers 120 and the outer shell 122 may be selected to maximize energy return during the gait cycle while in other situations one or more of the fillers 120 and the outer shell 122 may be designed to minimize rollover resistance.

Additionally, fillers 120 may also provide structural support for the footshell 112 during the gait cycle. For example, as shown in FIG. 1, the toe wedge 132 is sized and shaped to be inserted into a forefoot portion 138 of the footshell 112 to support the footshell 112 as the footshell 112 is inserted into a shoe (not shown). The shoe filler 136 is also inserted into the forefoot portion 138 of the footshell 112 to support the footshell 112 as the footshell 112 is inserted into the shoe. Additionally, the heel wedge 134 provides structural support for a heel portion 140 of the footshell 112 during the gait cycle. Accordingly, the fillers 120 may also provide structural support for the footshell 112 during the gait cycle. In some variants, the fillers may be made integrally with the footshell 112, manufactured as integral regions which may have different properties from the outer shell 122.

Additionally, an outer skin 142 and/or an inner skin 144 may be printed onto an external surface 146 and/or an internal surface 148 of one or more of the fillers 120 and/or the outer shell 122 resulting in a sandwich construction including a three-dimensional lattice core. The outer skin 142 may be thicker or thinner than the inner skin 144, and the outer skin 142 may be printed using a different material than the inner skin 144. Either of the thickness and material of the outer skin 142 and/or the inner skin 144 may vary locally to optimize weight, durability, performance, and/or other mechanical properties. The outer skin 142 and/or the inner skin 144 may be simultaneously printed with the lattice structure during the manufacturing process in a single manufacturing process. Perforations (not shown) may be included to facilitate cushioning and reduce weight. Custom color selections of the outer skin 142 and/or the inner skin 144 could be accommodated during the manufacturing process to mimic skin tones.

In the illustrated embodiment, the adapter 116 includes a pyramid adapter. In alternative embodiments, the adapter 116 may include any type of adapter that enables the 3D printed device 100 to operate as described herein. In the illustrated embodiment, the adapter 116 is attached to the spring 118. In alternative embodiments, the adapter 116 may be attached to any portion of the 3D printed device 100 that enables the 3D printed device 100 to operate as described herein.

In the illustrated embodiment, the spring 118 includes a leaf spring that includes a slender arc-shaped length of an elastic material having a rectangular cross-section. In alternative embodiments, the spring 118 may be any type of spring that enables the 3D printed device 100 to operate as described herein. Additionally, in the illustrated embodiment, the spring 118 may be formed of at least one of a metal, a laminated fiber reinforced composite material, a high performance thermoplastic material such as polyetherketone (PEK), polyetheretherketone (PEEK), polyaryletherketone (PAEK), polyimide (PI), poly-para-phenylene (PPP), polyphenylene sulfide (PPS), polyamideimide (PAI), polybenzimidazole (PBI), or any other elastic material. In alternative embodiments, the spring 118 may be formed of any material that enables the 3D printed device 100 to operate as described herein. Additionally, the spring 118 may include a plurality of springs 118.

During the manufacturing process, the 3D printed device 100 may be customized to the user's size, weight and activity level to provide a more comfortable user experience. Specifically, the material of construction of the footshell 112, the fillers 120, the adapter 116, the spring 118, and the skins 142 and 144 may be customized to the user's weight and activity level to provide a more comfortable user experience. For example, the materials of construction of each of the footshell 112, the fillers 120, the adapter 116, the spring 118, and the skins 142 and 144 may be customized to optimize at least one of the weight and the mechanical properties of the 3D printed device 100. The footshell 112, the fillers 120, the adapter 116, the spring 118, and the skins 142 and 144 are then assembled into the 3D printed device 100. In some embodiments, the footshell 112, the fillers 120, the adapter 116, the spring 118, and the skins 142 and 144 may be replaced such that the user may optimize the 3D printed device 100 to their weight and activity level. Accordingly, the 3D printed device 100 described herein may be customized during the manufacturing process and/or may be customized during use by the user based on the user's size, weight and activity level to provide a more comfortable user experience.

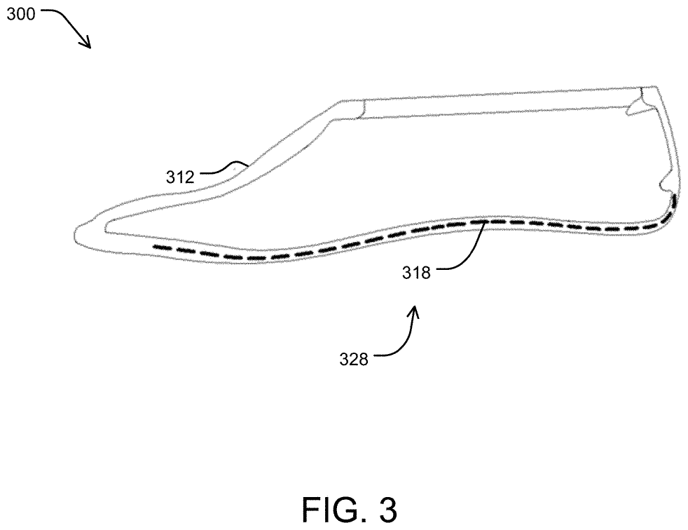

FIG. 3 illustrates a schematic cross section of an alternative embodiment of a footshell 312. In the embodiment illustrated in FIG. 3, the footshell 312 includes a spring 318 embedded within the footshell 312. Specifically, the footshell 312 is formed using the additive manufacturing processes described above and the spring 318 is embedded in a plantar portion 328 of the footshell 312. The spring 318 may be formed of the same material as the footshell 312 or may be formed of a different material. For example, the spring 318 may be formed of a composite fiber reinforced material. The footshell 312 may be manufactured by printing directly on a separately manufactured spring 318, by altering the density of the lattice structure in the plantar portion 328 of the footshell 312 using the same material, and/or by printing a multiple materials including a fiber reinforced material such as a unidirectional fiber reinforced layers to produce a laminated spring 318. The laminated spring 318 may be layers of unidirectional fiber oriented with different layers oriented in different directions. In an alternative embodiment, the footshell 312 may be manufactured to include a cavity to accept the spring 318. The spring 318 could be metal, a laminated fiber reinforced composite material, a high performance thermoplastic material such as PEK, PEEK, PAEK, PI, PPP, PPS, PAI, PBI, or any other elastic material. The spring 318 may extend the full length and width of the footshell 312 or it may be smaller in either length or width or both. The spring 318 may be a single spring 318 or a plurality of springs 318 positioned in a localized areas of the footshell 312.

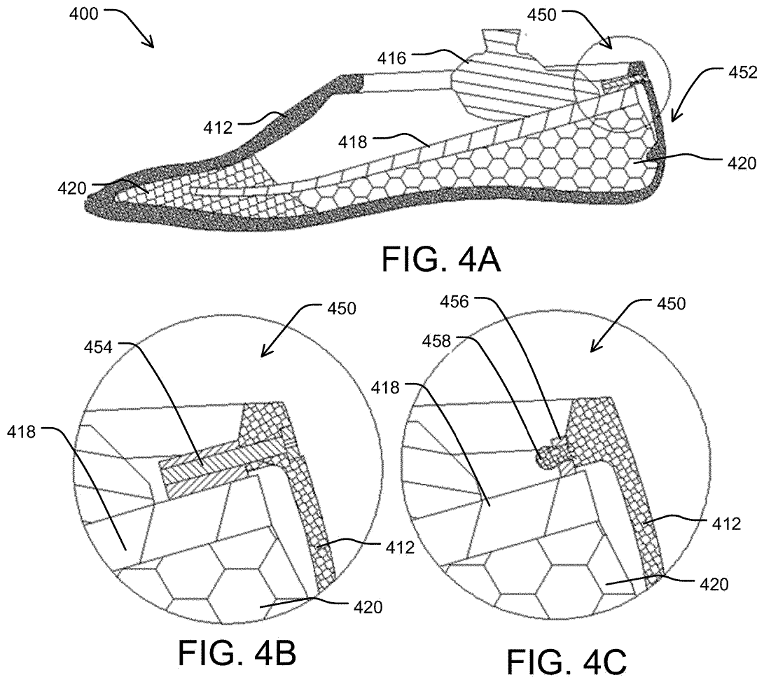

FIGS. 4A, 4B and 4C illustrate schematic cross sections of an alternative embodiment of a 3D printed device 400. The 3D printed device 400 includes a footshell 412, an adapter 416, a spring 418, one or more fillers 420, and a fastening mechanism 450. The fastening mechanism 450 secures the foot shell to the foot spring 418 or adapter 416 and enables the user to remove one or more of the adapter 416, the spring 418, and the one or more fillers 420 to enable the user to customize the 3D printed device 400 as described herein. In the illustrated embodiment, the fastening mechanism 450 is located in a heel portion 452 of the footshell 412 to secure the adapter 416, the spring 418, and the one or more fillers 420 in the footshell 412. In alternative embodiments, the fastening mechanism 450 may be located in any portion and attached to any component of the 3D printed device 400. The fastening mechanism 450 include threaded fasteners, spring pins, snaps, snap-fit fasteners, push-in fasteners, latches, straps, hook-n-loop fasteners, dial lacing systems, and/or any other type of fastener that enables the 3D printed device 400 to operate as described herein and which may be used to secure the adapter 416, the spring 418, and the one or more fillers 420 in the footshell 412. As shown in FIG. 4B, the fastening mechanism 450 may include a threaded fastener such as a screw 454. Alternatively, as shown in FIG. 4C, the fastening mechanism 450 includes a snap-fit fastener including a hook 456 and a pin 458. The fastening mechanism 450 enables the user to secure or remove the adapter 416, the spring 418, and the one or more fillers 420 from the footshell 412. Thus, the fastening mechanism 450 enables the user to customize the 3D printed device 400 as described herein.

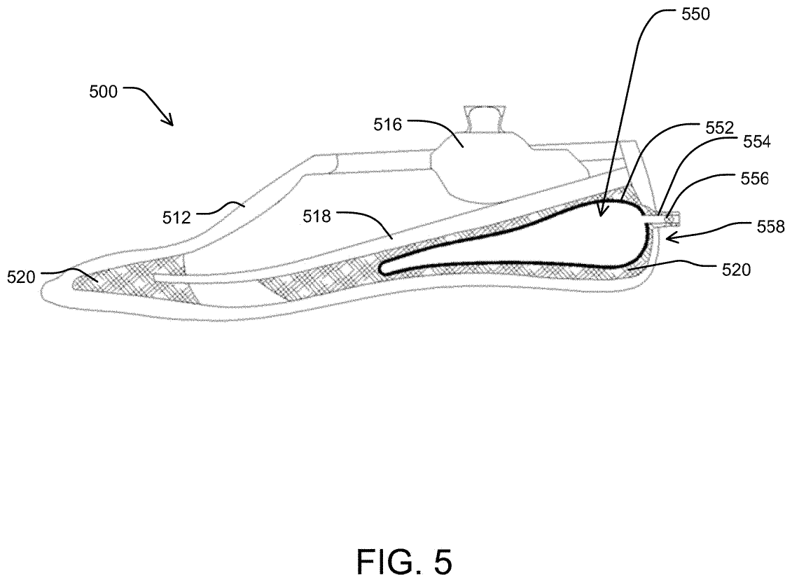

FIG. 5 illustrates a schematic cross section of an alternative embodiment of a 3D printed device 500. The 3D printed device 500 includes a footshell 512, an adapter 516, a spring 518, and one or more fillers 520. One of the fillers 520 defines a cavity 550 and a bladder 552 is positioned within the cavity 550. The bladder 552 includes an inlet 554 and a valve 556 that enables the bladder 552 to be selectively inflated and deflated to customize the stiffness of a heel portion 558 of the 3D printed device 500. The bladder 552 functions as a pneumatic spring or as a pump to customize the stiffness of the heel portion 558 of the 3D printed device 500. Adjusting or tuning the pressure in the bladder 552 adjusts or tunes the stiffness of the heel portion 558 and allows the 3D printed device 500 to be optimized for the user. The bladder 552 may have a plurality of valves 556 and the valves 556 may be check valves such that the cavity 550 and the valves 556 act as a pump. The bladder 552 may include an internal lattice or a porous rib structure to that restore or expand the bladder 552 after compression without inhibiting pump function. The bladder 552 may be printed integrally with the filler 520 or the footshell 512 or may be a cavity which allows for a separate bladder 552.

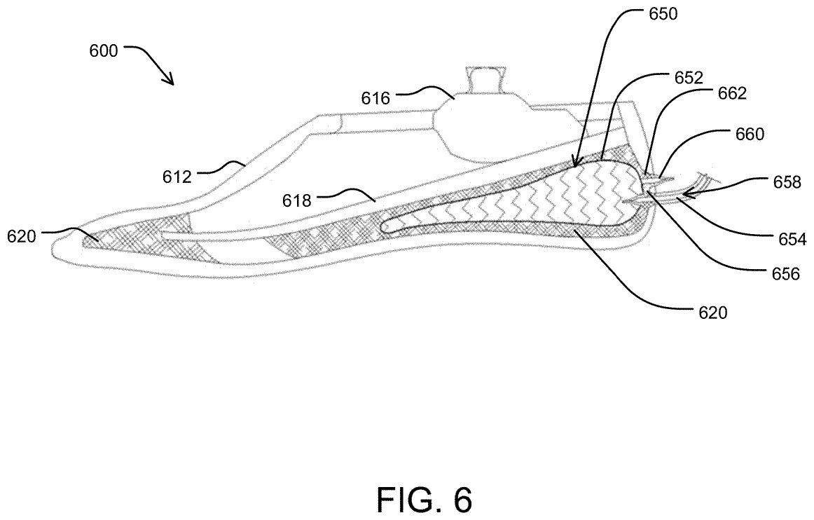

FIG. 6 illustrates a schematic cross section of an alternative embodiment of a 3D printed device 600. The 3D printed device 600 includes a footshell 612, an adapter 616, a spring 618, and one or more fillers 620. The 3D printed device 600 is substantially similar to the 3D printed device 500 because one of the fillers 620 defines a cavity 650 and a bladder 652 is positioned within the cavity 650. The bladder 652 includes an inlet 654, an inlet valve 656, an outlet 660, and an outlet valve 662 that enables the bladder 652 to be selectively inflated and deflated to customize the stiffness of a heel portion 658 of the 3D printed device 600. The bladder 652 functions as a pneumatic spring to customize the stiffness of the heel portion 658 of the 3D printed device 600, or as a pump to evacuate air from a prosthetic socket. In the illustrated embodiment, the inlet valve 656 and the outlet valve 662 are check valves or one-way valves that enable selective inflation and deflation of the bladder 652 to customize the stiffness of a heel portion 658 of the 3D printed device 600. In the illustrated embodiment, the bladder 652 includes two one-way valves 656 and 662 that expel air from the bladder 652 and direct the air to a location. The location may be the atmosphere or another portion of the 3D printed device 600. The bladder 652 expands as weight is removed from the heel portion 658 and the bladder 658 deflates as weight is added to the heel portion 658. As the bladder expands it may draw air in from a prosthetic socket, resulting in a vacuum condition within the socket to provide vacuum socket suspension.

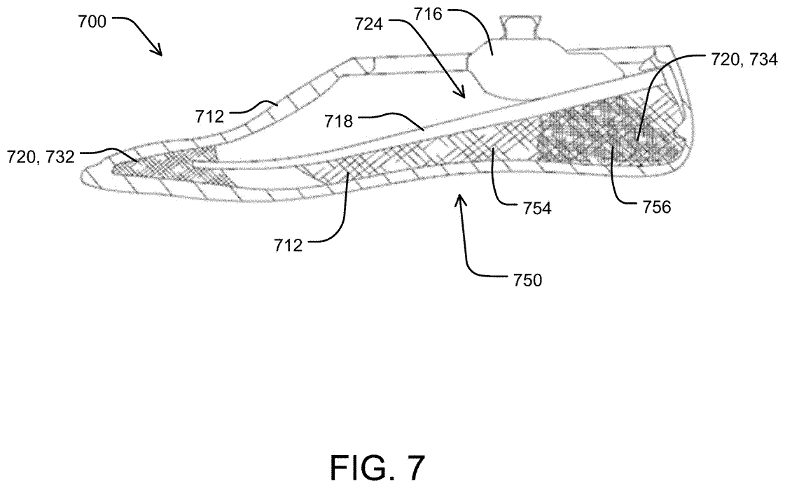

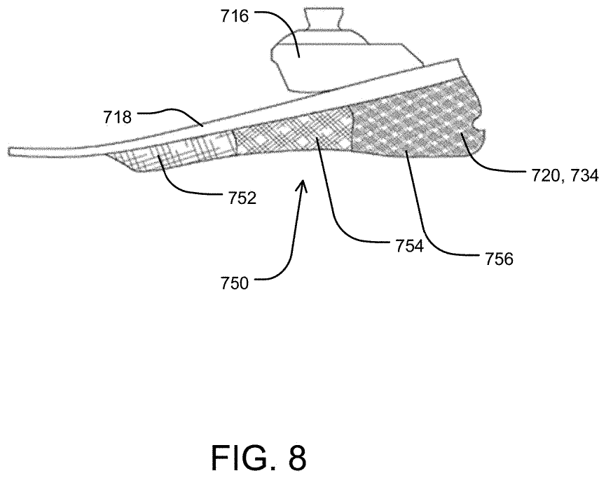

FIG. 7 illustrates a schematic cross section of an alternative embodiment of a 3D printed device 700. FIG. 8 illustrates a schematic cross section of a portion of the 3D printed device 700. The 3D printed device 700 includes a footshell 712, an adapter 716, a spring 718, and one or more fillers 720. Specifically, the fillers 720 include a toe wedge 732 and a heel wedge 734 arranged with the adapter 716 and the spring 718 in a footshell cavity 724 defined by the footshell 712. In the illustrated embodiment, the heel wedge 734 includes a plurality of sections 750 that are additive manufactured with different materials, different densities, and/or different lattice structures to enable the 3D printed device 700 to be customized to optimize at least one of the weight and the mechanical properties of the 3D printed device 700. For example, in the illustrated embodiment, the heel wedge 734 includes a first section 752, a second section 754, and a third section 756. In some embodiments, the first, second, and third sections 752-756 as well as the toe wedge 732 may be additively manufactured with different lattice structures to tune or optimize the stiffness properties of the 3D printed device 700 for different activity levels of the user. In another embodiment, the first, second, and third sections 752-756 as well as the toe wedge 732 may be additively manufactured with different materials that have different such that some sections 752-756 are harder or softer than other sections 752-756. Varying the hardness enables the 3D printed device 700 to be optimized for different activity levels of the user. Moreover, the sections 752-756 may be customized to optimize performance of the 3D printed device 700 during different phases of the gait cycle. For example, the first section 752 may be additively manufactured with a material that optimizes the performance of the 3D printed device 700 during the propulsion phase of the gait cycle. Similarly, the second section 754 may be additively manufactured with a material that optimizes the performance of the 3D printed device 700 during the roll over phase of the gait cycle and the third section 756 may be additively manufactured with a material that optimizes the performance of the 3D printed device 700 during the heel strike phase of the gait cycle.

Varying the stiffness of different sections 752-756 in the heel wedge 734 enables optimization of the performance behavior of the 3D printed device 700. For example, varying the compression characteristics of the 3D printed device 700 located under the distalmost structural component will alter foot performance. Specifically, softening the compression characteristics of the anterior and posterior ends of the 3D printed device 700 relative to the midfoot or arch of the 3D printed device 700 decreases the resistance and effort required to roll over the 3D printed device 700. Stiffening the anterior and posterior sections of the 3D printed device 700 relative to the midfoot or arch sections of the 3D printed device 700 increase the ankle moment required for roll over and over the effort require for rollover. Properties of the lattice structure may also be altered in the medial-laterial direction to allow inversion and eversion and more accurately replicate human foot and ankle behavior. 3D printed devices 700 exhibiting different stiffness characteristics in different areas or zones of the 3D printed device 700 may allow a single foot to be utilized for users of different activity levels, or the same user as the user's activity level increases or decreases, without replacing or exchanging the structural components of the 3D printed device 700. The performance of the 3D printed device 700 may be altered by changing footshells 712 and/or fillers 720, for example as a user recovers from or suffers an injury. A customized 3D printed device 700 may be used to create a controlled stiffness gradient for either an individual user or for a class of users based on activity level. Optimizing the roll over behavior of the 3D printed device 700 may be achieved by altering the characteristics of the footshell 712 and/or fillers 720.

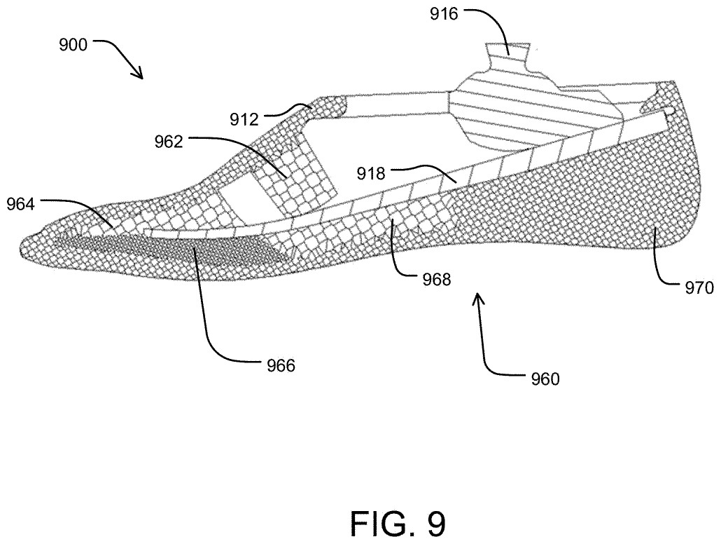

FIG. 9 illustrates a schematic cross section of an alternative embodiment of a 3D printed device 900. The 3D printed device 900 includes a footshell 912, an adapter 916, and a spring 918. The 3D printed device 900 is substantially similar to the 3D printed device 700 except that the fillers 720 have been incorporated into the footshell 912 during the manufacturing process. Specifically, the footshell 912 is additively manufactured such that the footshell 912 includes a plurality of zones 960 that are additive manufactured with different materials, different densities, and/or different lattice structures to enable the 3D printed device 900 to be customized to optimize at least one of the weight and the mechanical properties of the 3D printed device 900. For example, in the illustrated embodiment, the footshell 912 includes a first zone 962, a second zone 964, a third zone 966, a fourth zone 968, and a fifth zone 970. In the illustrated embodiment, the first-fifth zones 962-970 are additively manufactured with the footshell 912 with different lattice structures to tune or optimize the stiffness properties of the 3D printed device 900 for different activity levels of the user. In another embodiment, the first-fifth zones 962-970 are additively manufactured with the footshell 912 with different materials that have different hardnesses such that some zones 962-970 are harder or softer than other zones 962-970. Varying the hardness enables the 3D printed device 900 to be optimized for different activity levels of the user.

Varying the lattice design of the zones 962-970 minimizes weight and optimizes performance while providing stiffness and durability in localized areas of the 3D printed device 900. For example, the illustrated embodiment enables the 3D printed device 900 to be inserted into a shoe and enables the shoelaces of the shoe to be tightened without collapsing or compressing the footshell 912. Additionally, varying the lattice design of the zones 962-970 may also optimize the stiffness for users of different weights and activity levels. 3D printed devices 900 with different properties can be interchanged as a user recovers from surgery or a temporary residual limb injury and the user's actability level increases. Rather than purchasing a new foot to accommodate increased or decreased activity level, utilizing more than one 3D printed device 900 can alter the foot performance to accommodate the user's needs.

In alternative embodiments, 3D printed devices 100-900 may also include integrated supports, cushioning areas, and/or bladders that may be printed directly into and onto the structural components of the footshells 112-912 of the 3D printed devices 100-900. Thus, the footshells 112-912 are securely attached to other structural components of the 3D printed devices 100-900, providing a firm attachment and enabling a high level of performance during athletic activities. Additionally, it may also be possible to remove the footshells 112-912 from the 3D printed devices 100-900 to replace the footshells 112-912 with other footshells 112-912 that are better suited to other tasks.

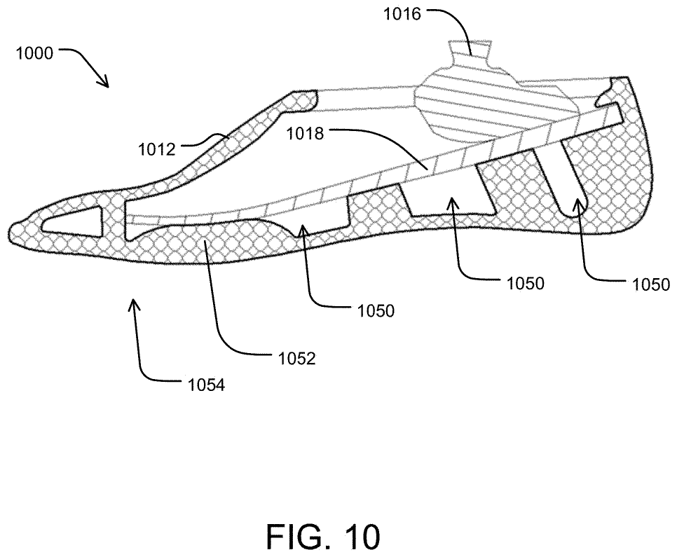

FIG. 10 illustrates a schematic cross section of an alternative embodiment of a 3D printed device 1000. The 3D printed device 1000 includes a footshell 1012, an adapter 1016, and a spring 1018. The 3D printed device 1000 is substantially similar to the 3D printed device 900 except that the voids 1050 have been incorporated into the footshell 1012 during the manufacturing process. In the illustrated embodiment, the footshell 1012 is printed as a single unit in a single manufacturing operation and is additively manufactured to include voids 1050 which limit the contact area of the spring 1018 and the footshell 1012. The voids 1050 reduce the weight of the 3D printed device 1000 and alter the rollover performance of the 3D printed device 1000. Specifically, the size, shape and location of the voids 1050 may be designed to optimize rollover behavior of the 3D printed device 1000. For example, in the illustrated embodiment, the footshell 1012 includes voids 1050 that define a curved contact area 1052 located at a toe end 1054 of the spring 1018 that alters the pressure distribution on the spring 1018 when pressure is on a forefoot portion 1056 of the 3D printed device 1000 and may alter the center of pressure, which may change the function of the 3D printed device 1000. Thus, the voids 1050 may alter the pressure distribution on the spring 1018 within the 3D printed device 1000, optimizing the performance characteristics of the 3D printed device 1000.

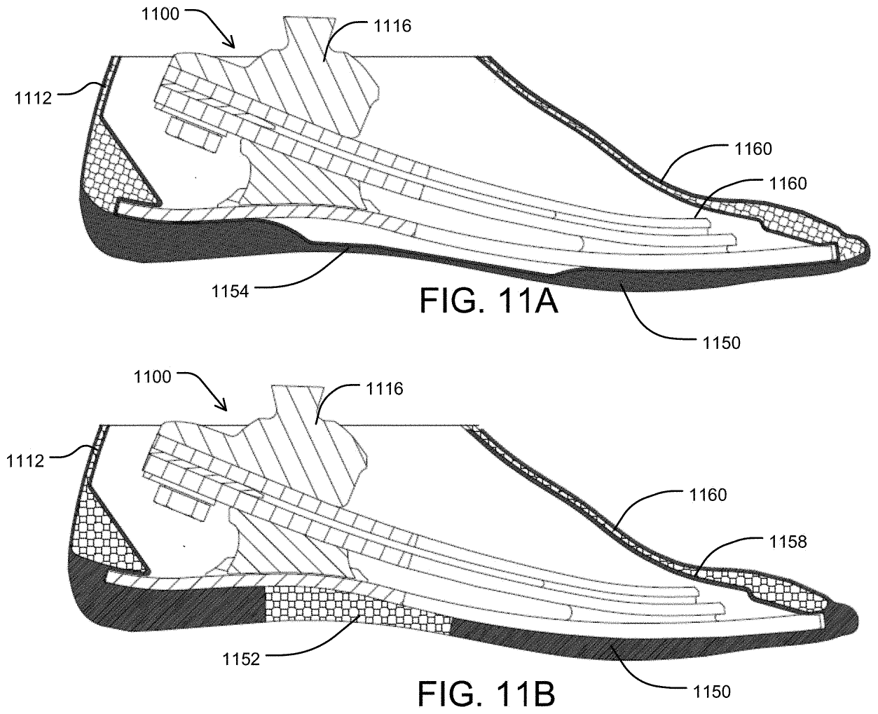

FIG. 11A illustrates a schematic cross section of an alternative embodiment of a 3D printed device 1102. FIG. 11B illustrates a schematic cross section of the 3D printed device 1150. The 3D printed devices 1100 and 1102 include a footshell 1112, an adapter 1116, and a sole 1150. Specifically, in the illustrated embodiment, the sole 1150 includes a wear resistant, high friction sole surface integrated into the footshell 1112 that provides increased durability and function during the gait cycle. The sole 1150 is additively manufactured with the footshell 1112 such that the sole 1150 is integrally formed with the footshell 1112, decreasing manufacturing costs and increasing the durability of the 3D printed device 1100. In some embodiments, the sole 1150 may be a solid material without voids or lattice structure. The portion of the footshell 1112 located beneath the distal structural component or components of a prosthetic foot are subjected to repeated compressing loads during walking. Because footshells are commonly made of elastomeric materials, which exhibit creep behavior and limited strength, lattice structures located beneath the distal structural components of a prosthetic foot may experience deformation and may be crushed over time. Footshells, such as footshell 1112, that include solid sole material 1150 covering at least 30% of the distal surface area of the distal structural components of the footshell 1112 such that the footshell 1112 have improved durability. Specifically, lattice layers are typically more brittle than solid sole material 1150 and may chip or break more easily than the solid sole material 1150. In the illustrated embodiment, the footshell 1112 includes the solid sole material 1150 covering at least 30% of the distal surface area of the distal structural component or components of the foot such that the footshell 1112 has improved durability. A footshell with 30-100% of the distal surface of the foot covered by solid sole material combined with lattice structures in low stress areas of the footshell results in an optimum combination of durability and reduced weight.

Depending on the shape of the 3D printed device 1102, the arch area of the footshell 1112 may experience reduced pressures compared to the heel and forefoot areas, and hence a lattice 1152 located in the arch section may result in reduced weight. Alternatively, an area of thin solid material 1154 located in the distal arch or midfoot area may achieve the same or similar weight reduction and require less manufacturing time than the lattice structure 1152. Solid elastomers have a density of 1.0 g/cc or higher. An upper section 1158 of the footshell 1112may be formed of a lattice structure. The upper section 1158 may be formed of a lattice structure with a skin or layer of solid material 1160 on the outer surface. The upper section 1158 may be formed of a lattice sandwich structure comprised of a lattice core having skins on both the exterior and interior surfaces of the footshell. A skin 1160 on the exterior surface prevents dirt, debris, and water from infiltrating the lattice structure and also prevent the lattice structure from getting snagged and broken. A skin on the inner surface may facilities insertion of structural foot components into a shoe and increases durability by decreasing the amount of wear between structural foot components and the upper section of the footshell.

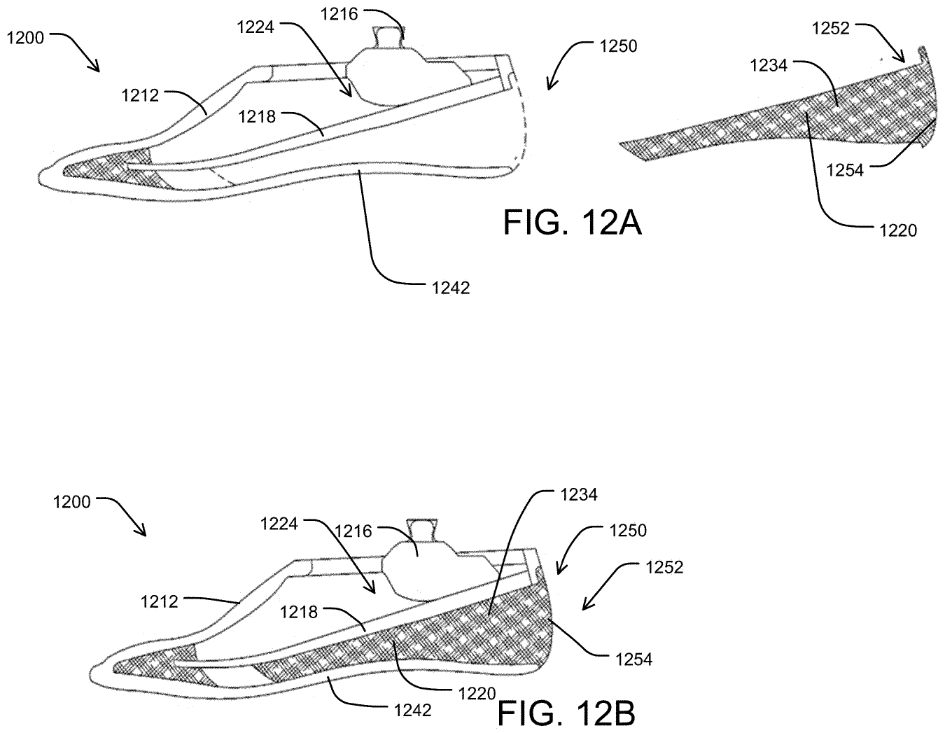

FIG. 12A illustrates a schematic cross section of an alternative embodiment of a 3D printed device 1200 in a first configuration. FIG. 12B illustrates a schematic cross section of the 3D printed device 1200 in a second configuration. The 3D printed device 1200 includes a footshell 1212, an adapter 1216, a spring 1218, and at least one filler 1220. In the illustrated embodiment, at least one of the fillers 1220 are selectively removable from the 3D printed device 1200 to enable the 3D printed device 1200 to be customized. Specifically, the footshell 1212 defines a footshell cavity 1224 and an opening 1250 and the fillers 1220 includes a heel wedge 1234. The heel wedge 1234 is selectively insertable into the footshell cavity 1224 through the opening 1250 to enabled immediate and easy access to the heel wedge 1234 to enable the 3D printed device 1200 to be customized to the activity level of the user. Additionally, a posterior end 1252 of the heel wedge 1234 has an outer skin 1254 that may be manufactured to match an outer skin 1242 of the footshell 1212, if present.

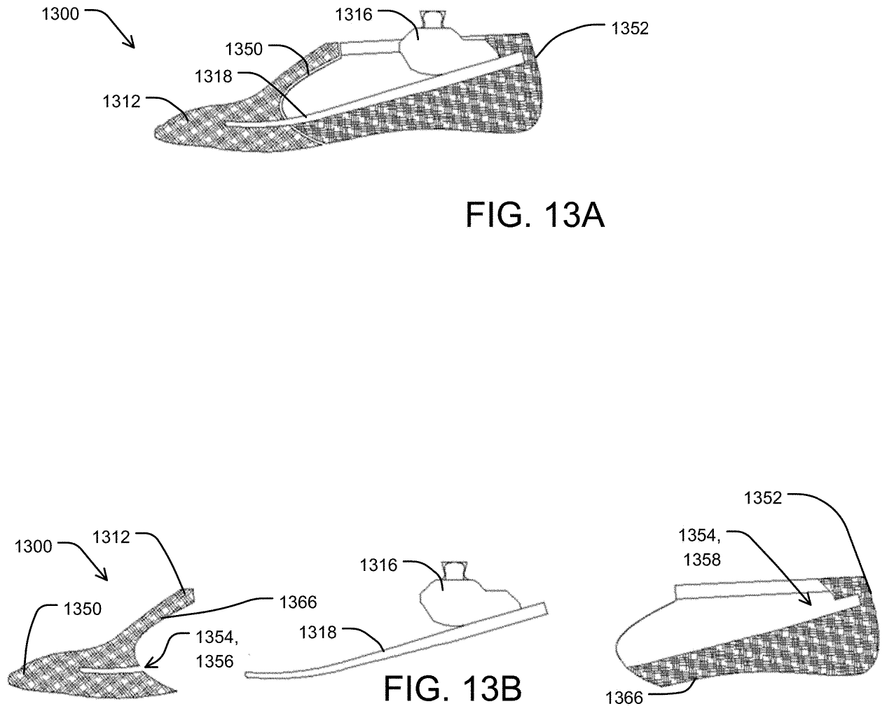

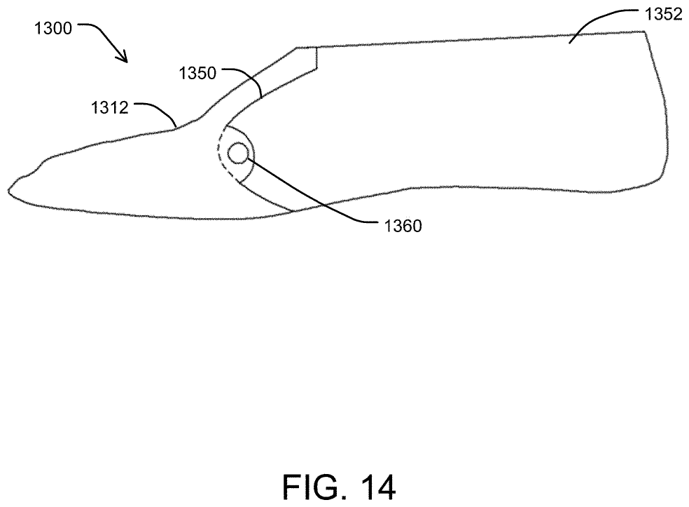

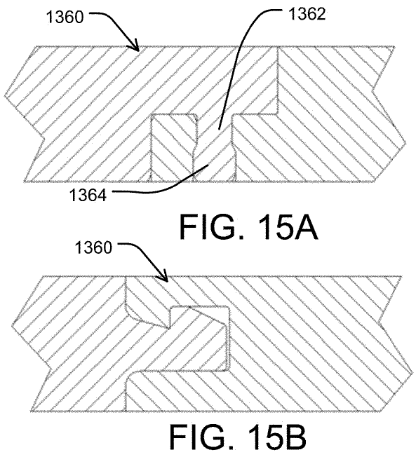

FIG. 13A illustrates a schematic cross section of an alternative embodiment of a 3D printed device 1300 in a first configuration. FIG. 13B illustrates a schematic cross section of the 3D printed device 1300 in a second, disassembled configuration. FIG. 14 illustrates another schematic cross section of the 3D printed device 1300. FIGS. 15A-B illustrate another schematic cross section of a portion of the 3D printed device 1300. The 3D printed device 1300 includes a footshell 1312, an adapter 1316, and a spring 1318. In the illustrated embodiment, the footshell 1312 is split into a first portion 1350 and a second portion 1352. In the illustrated embodiment, the first portion 1350 is a toe section or an anterior of the footshell 1312 and the second portion 1352 is a heel section or a posterior of the footshell 1352. The adapter 1316 is attached to the spring 1318 and the first and second portions 1350 and 1352 are additively manufactured to include voids 1354 that correspond to the size and shape of the adapter 1316 and the spring 1318. Specifically, the voids 1354 include a first portion void 1356 and a second portion void 1358. The first portion void 1356 is configured to receive an anterior portion of the spring 1318 and the second portion void 1358 is configured to receive a posterior portion of the spring 1318 and the adapter 1316. During operations, the 3D printed device 1300 is assembled by sliding the spring 1318 into the first portion void 1356 and sliding the posterior portion of the spring 1318 and the adapter 1316 into the second portion void 1358 such that the first portion 1350 and the second portion 1352 form the footshell 1312.

As shown in FIGS. 14, 15A, and 15B, the 3D printed device 1300 may include a connector 1360 that attaches the first portion 1350 to the second portion 1352 when the first and second portions 1350 and 1352 are joined to form the footshell 1312. The connector 1360 may include a snap, a hook and loop fastener, magnets, and/or any other attachment mechanism that attaches the first portion 1350 to the second portion 1352. For example, FIG. 15A illustrates the connector 1360 as a snap including interlocking features that interlock together to attach the first portion 1350 to the second portion 1352. The interlocking features include a protrusion 1362 that is configured to be selectively inserted and extracted into and out of a hole 1364 to attach the first portion 1350 to the second portion 1352. The protrusion 1362 may have a round, oblong, and/or rectangular shape. Another embodiment illustrated in FIG. 15B illustrates the connector 1360 including interlocking features which may be continuous around a perimeter or have discrete, intermittent interlocking features along mating surfaces. Additionally, edges 1366 of the first portion 1350 to the second portion 1352 may include a joint and/or interface that attaches the first portion 1350 to the second portion 1352 or guides the first portion 1350 and the second portion 1352 together to enable the connector 1360 to attach the first portion 1350 to the second portion

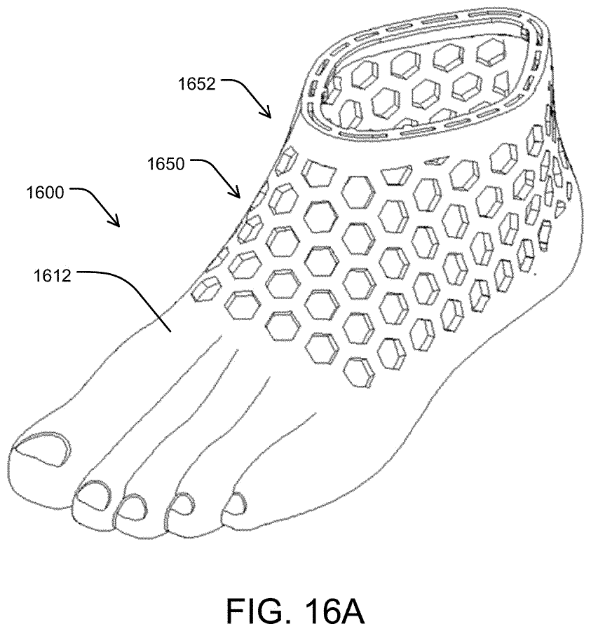

FIG. 16A illustrates a perspective view of an alternative embodiment of a 3D printed device 1600 including a footshell 1612. FIG. 16B illustrates a cross-sectional view of the footshell 1612. In the illustrated embodiment, the footshell 1612 is additively manufactured to include a pattern of cutouts or perforations 1650 extending through the footshell 1612 which may reduce the amount of material used to manufacture the footshell 1612 and reduce the overall weight of the footshell 1612. Repeating ordered perforations through a wall or shell is referred as a 2D lattice. In the illustrated embodiment, the pattern of cutouts 1650 includes a pattern of hexagonal cutouts incorporated into an upper portion 1652 of the footshell 1612. In alternative embodiments, the pattern of cutouts 1650 may include a pattern of round, oval, or triangular cutouts. In alternative embodiments, the pattern of cutouts may be a pattern of variably shaped cutouts. In other embodiments, the cutouts may have any shape that enables the footshell 1612 to operate as described herein. The 3D printed device 1600 may also include an outer shell 1622, an outer skin 1642 and an inner skin 1644 to form a sandwich construction. In the illustrated embodiment, the outer skin 1642, the inner skin 1644, and the footshell 1612 vary such that the thickness of the 3D printed device 1600 varies from about 3 millimeters (mm) to about 15 mm. The 3D printed device 1600 including the pattern of cutouts 1650 in addition to a sandwich wall construction reduces a weight of the footshell 1312 by about 30% to about 40% while increasing the durability (as measured during ISO 22675 testing) of the footshell 1612 and the 3D printed device 1600. In one example, the sandwich wall had a 1mm outer skin thickness and a 0.6 mm inner skin thickness with a lattice core. The footshell was printed on a Snapmaker printer using ESUN eLastic TPE-85A material. The weight reduction was 32% and the device exhibited increased durability as compared to a current commercially available footshell.

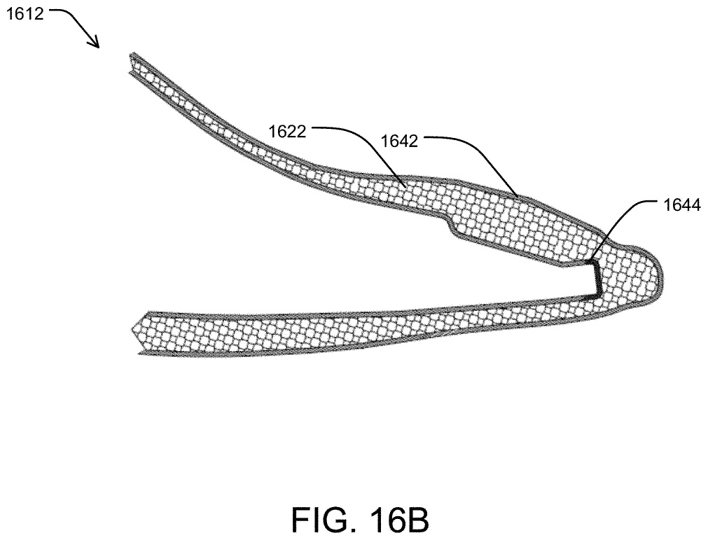

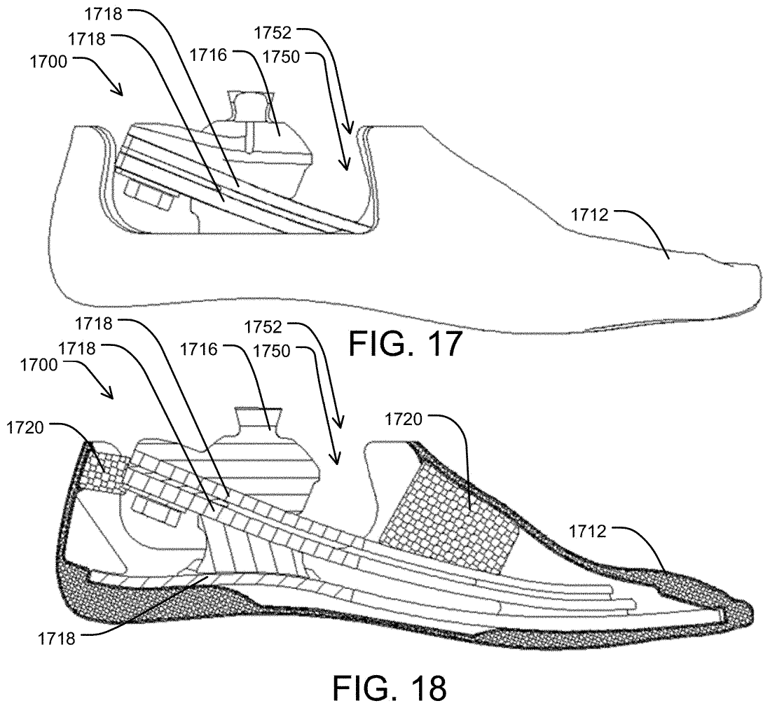

FIG. 17 illustrates a side view of an alternative embodiment of a 3D printed device 1700 including a footshell 1712. FIG. 18 illustrates a schematic cross section of the 3D printed device 1700. As shown in FIGS. 17 and 18, the 3D printed device 1700 includes the footshell 1712, an adapter 1716, at least one spring 1718, and at least one filler 1720. An anterior filler 1720 located in the forefoot region and/or a posterior filler 1720 located in a heel region may prevent the footshell from collapsing when shoe laces or other shoe retaining mechanisms are tightened, and in general improve the fit between the footshell and the structural components of the prosthetic foot, improving performance of the prosthetic foot and footshell combination by reducing sliding between the two in the medial-lateral and anterior-posterior directions. Additionally, the footshell 1712 defines a low-cut lateral cutout 1750 and a low-cut medial cutout 1752 that reduce the weight of the footshell 1712. Additionally, the at least one spring 1718 includes a plurality of springs 1718 and the at least one filler 1720 includes a plurality of fillers 1720 that are sized and shaped to conform to and support the plurality of springs 1718.

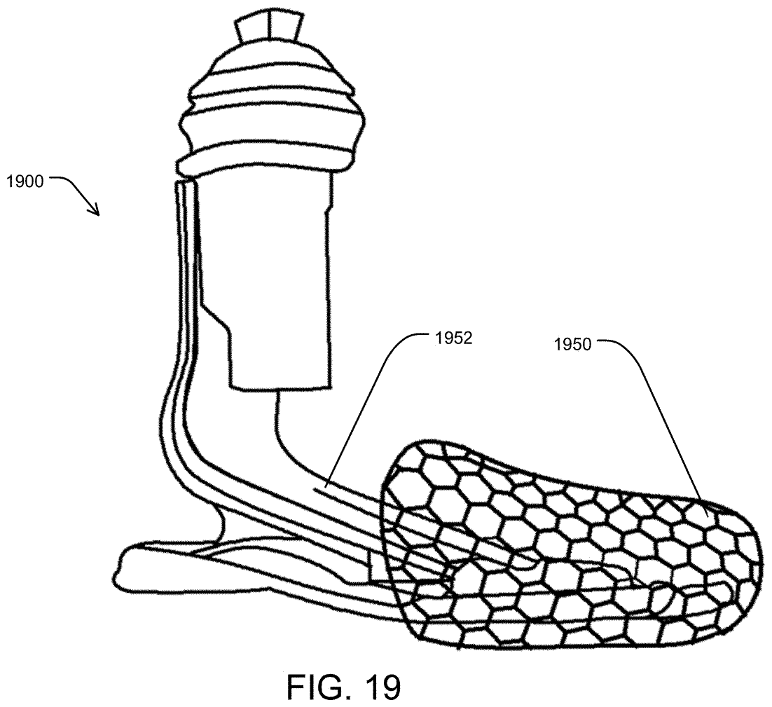

FIG. 19 illustrates a perspective view of an alternative embodiment of a 3D printed device 1900 including a shoe filler 1950. In the illustrated embodiment, the shoe filler 1950 is located between a prosthetic foot 1952 and the footshell (not shown) in the forefoot area and prevents the footshell from collapsing when shoelaces are tightened. The shoe filler 1950 is manufacturing using an additive manufacturing process and is formed of a lattice structure as described herein to enable the properties of the shoe filler 1950 to be optimized for the activity level of the user. The shoe filler may be construction of a common, non-elastomeric thermoplastic material such as polyamid and is formed as a shell structure with through-the-thickness cutouts or perforations, (i.e. a 2D lattice).

FIG. 20 illustrates atomic cell structures which may be used as fundamental building blocks for many of the lattice structures that could be used with the 3D printed devices disclosed herein. The cell structures show in FIG. 20 can mimic naturally occurring atomic structures such as cubic, tetragonal, orthorhombic, rhombohedral, monoclinic, triclinic, including body centered, face centered, and base centered variations of these atomic cells.

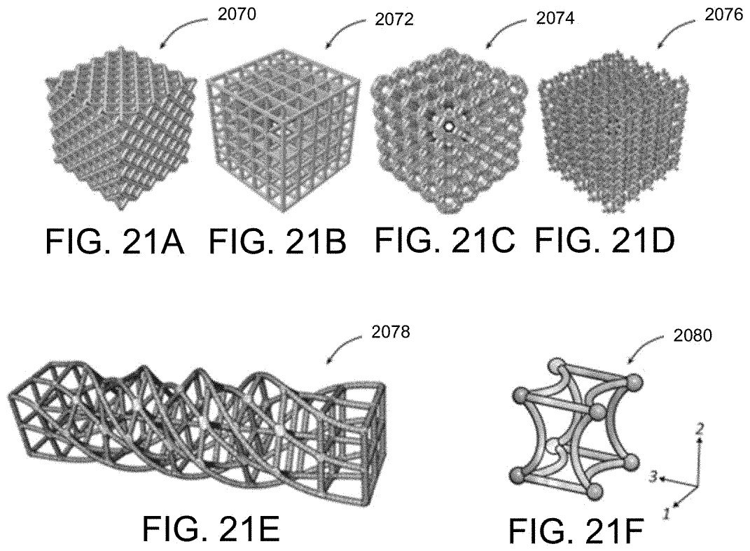

FIGS. 21A-21F illustrate various lattice structures that may be possible for use with the 3D printed devices disclosed herein. Each of the lattice structures 2070 (FIG. 21A), 2072 (FIG. 21B), 2074 (FIG. 21C), 2076 (FIG. 21D), 2078 (FIG. 21E), and 2080 (FIG. 21F) have unique shapes, sizes, and orientations for the individual strut members of the lattice structure as well as the resulting shapes and other features of the lattice structure as a whole. The various lattice structures shown in FIGS. 21A-21F may each provide different properties such as strength, compressibility, flexibility, elasticity, resistance to torque, etc. FIG. 21F shows an example of a lattice unit cell utilizing both semi-circular and straight beams between unit cell connection points. Because the curved beams are not as stiff as the straight beams, the cell is stiffer in compression in Direction 3 than and in Directions 1 and 2. When the straight beams are compressed in Direction 1, the straight beams will demonstrate a high stiffness until buckling occurs, at which point the stiffness will decrease dramatically. The behavior of the curved beams, when compressed in either Direction 1 or 2, do not exhibit buckling behavior. The curved beams are pre-buckled by their semi-circular shape.

The examples shown in FIG. 21A-21F are exemplary only of the infinite number of lattice structure designs that are possible. The lattice designs that are used for various portions of any of the 3D printed devices disclosed herein may be optimized for use as footshells, exterior protective surfaces, and other features of a 3D printed device that is used and/or capable of being used with a limb such as a residual limb of a user. The various lattice structures disclosed herein may provide certain advantages as compared to other types of materials, for example solid or foam material, such as the ability to integrally form a footshell with regions of different mechanical properties.

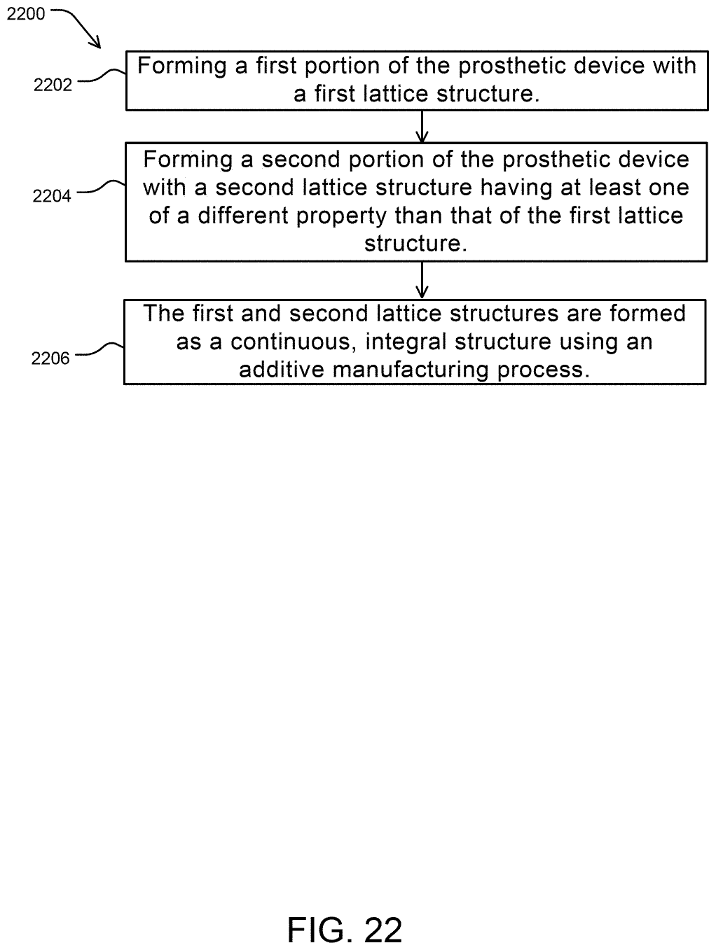

FIG. 22 is a flow diagram illustrating an example method of forming a footshell. A first step of a method 2200 includes forming 2202 a first portion of the prosthetic device with a first lattice structure. A second step 2204 may include forming a second portion of the prosthetic device with a second lattice structure having at least one different property than that of the first lattice structure. A step 2206 includes forming the first and second lattice structures as a continuous, integral structure using an additive manufacturing process. The at least one different property may include at least one of lattice density, material composition, lattice structure, compressibility, porosity, and rigidity. The first portion may be a layer of a bottom of a footshell, and the second portion may be a layer of a top of the footshell where the layers are formed as a continuous, integral structure. Step 2206 may be iterated to produce multiple regions with differing properties. “Forming” may include any step in the manufacturing process including, without limitation, printing, designing, planning, manufacturing, molding, transporting, and/or any other step in the manufacturing process.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.” In addition, the term “based on” as used in the specification and the claims is to be construed as meaning “based at least upon.”

What is claimed is:

1. A prosthetic device, comprising:
a footshell comprising:
a first portion and a second portion, the first portion having a first portion polymer lattice structure and the second portion having a second portion polymer lattice structure, wherein the first portion polymer lattice structure has a first set of mechanical properties, and the second portion polymer lattice structure has a second set of mechanical properties different than the first set of mechanical properties; and
a sole attached to a bottom of the first portion and the second portion, wherein the sole is formed of a solid material.

2. The prosthetic device of claim 1, wherein the sole comprises a wear resistant, high friction sole surface.

3. The prosthetic device of claim 2, wherein the wear resistant, high friction sole surface is integrated into the first portion and the second portion.

4. The prosthetic device of claim 2, wherein the wear resistant, high friction sole surface is additively manufactured with the first portion and the second portion.

5. The prosthetic device of claim 1, wherein the sole comprises a lattice structure within an arch section of the footshell.

6. The prosthetic device of claim 1, wherein at least 10% of the solid material comprises a solid material having a density greater than 1.0 g/cc.

7. The prosthetic device of claim 1, wherein the first portion has a first density and the second portion has a second density different than the first density.

8. The prosthetic device of claim 1, wherein the first and second polymer lattice structures have a continuous, single-piece construction.

9. The prosthetic device of claim 1, wherein the first portion has a first stiffness and the second portion has a second stiffness different than the first stiffness.

10. The prosthetic device of claim 1, wherein the first portion has a first thickness and the second portion has a second thickness different than the first thickness.

11. A prosthetic device, comprising:
a footshell comprising:
a first portion and a second portion, the first portion having a first portion polymer lattice structure and the second portion is formed of a solid material,
wherein the second portion is a sole portion and the sole portion is located below a distalmost surface of a prosthetic foot.

12. The prosthetic device of claim 11, wherein the sole comprises a wear resistant, high friction sole surface.

13. The prosthetic device of claim 12, wherein the wear resistant, high friction sole surface is additively manufactured with the first portion.

14. The prosthetic device of claim 11, wherein the sole comprises a lattice structure within an arch section of the footshell.

15. The prosthetic device of claim 11, wherein the sole portion has a density greater than 1.0 g/cc.

16. The prosthetic device of claim 11, wherein the first portion has a first density and the second portion has a second density different than the first density.

17. The prosthetic device of claim 11, wherein the first and second portions have a continuous, single-piece construction.

18. The prosthetic device of claim 11, wherein the first portion has a first stiffness and the second portion has a second stiffness different than the first stiffness.

19. The prosthetic device of claim 11, wherein the first portion is fabricated using a material with a different chemical composition than the second portion.

20. The prosthetic device of claim 15, wherein the sole portion covers at least 30% of the distal surface of a prosthetic foot.
